SEQUENCE LISTING (35 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 33 aa, Homo sapiens
HADGSFSDEMNTILDNLAARDFINWLIQTKITD

SEQ ID NO: 3 - protein, 34 aa, Artificial Sequence
  GIP, GLP-1 or GLP-2 variants
  PEPTIDE (1)..(34): GLP-2
HADGSFSDEMNTILDNLAARDFINWLIQTKITDR

SEQ ID NO: 4 - protein, 30 aa, Homo sapiens
YAEGTFISDYSIAMDKIHQQDFVNWLLAQK

SEQ ID NO: 5 - protein, 33 aa, Artificial Sequence
  GIP, GLP-1 or GLP-2 variants
  PEPTIDE (1)..(33): GLP-2
HGDGSFSDEMNTILDNLAARDFINWLIQTKITD

SEQ ID NO: 6 - protein, 33 aa, Homo sapiens
HGDGSFSDEMNTILDNLAARDFINWLIETKITD

SEQ ID NO: 7 - protein, 37 aa, Artificial Sequence
  GIP, GLP-1 or GLP-2 variants
  PEPTIDE (1)..(37): hGLP-1
HDEFERHAEGTFTSDVSSYLEGQAAKEFIAWLVKGRG

SEQ ID NO: 8 - protein, 30 aa, Artificial Sequence
  GIP, GLP-1 or GLP-2 variants
  PEPTIDE (1)..(30): hGLP-1
HAEGTFTSDVSSYLEGQAAKEFIAWLVKGR

SEQ ID NO: 9 - protein, 31 aa, Artificial Sequence
  GIP, GLP-1 OR GLP-2 fragments
  PEPTIDE (1)..(31): hGLP-1
HAEGTFTSDVSSYLEGQAAKEFIAWLVKGRG

SEQ ID NO: 10 - protein, 36 aa, Artificial Sequence
  GIP, GLP-1 or GLP-2 variants
  PEPTIDE (1)..(36): hGLP-1
HDEFERHAEGTFTSDVSSYLEGQAAKEFIAWLVKGR

SEQ ID NO: 11 - protein, 28 aa, Artificial Sequence
  GIP, GLP-1 or GLP-2 variants
  PEPTIDE (1)..(28): hGLP-1
EGTFTSDVSSYLEGQAAKEFIAWLVKGR

SEQ ID NO: 12 - protein, 30 aa, Artificial Sequence
  GIP, GLP-1 or GLP-2 variants
  PEPTIDE (1)..(30): hGLP-1
AAEGTFTSDVSSYLEGQAAKEFIAWLVKGR

SEQ ID NO: 13 - protein, 30 aa, Artificial Sequence
  GIP, GLP-1 or GLP-2 variants
  PEPTIDE (1)..(30): hGLP-1
HAEATFTSDVSSYLEGQAAKEFIAWLVKGR

SEQ ID NO: 14 - protein, 29 aa, Artificial Sequence
  GIP, GLP-1 or GLP-2 variants
  PEPTIDE (1)..(29): GIP
AEGTFISDYSIAMDKIHQQDFVNWLLAQK

SEQ ID NO: 15 - protein, 42 aa, Artificial Sequence; identical to SEQ ID NO: 1
  GIP, GLP-1 or GLP-2 variants
  PEPTIDE (2)..(2): D-Ala

SEQ ID NO: 16 - protein, 42 aa, Artificial Sequence
  GIP, GLP-1 or GLP-2 variants
  PEPTIDE (1)..(42): GIP
YAPGTFISDYSIAMDKIHQQDFVNWLLAQKGKKNDWKHNITQ

SEQ ID NO: 17 - protein, 30 aa, Artificial Sequence; identical to SEQ ID NO: 4
  GIP, GLP-1 or GLP-2 variants
  PEPTIDE (2)..(2): D-Ala

SEQ ID NO: 18 - protein, 9 aa, Artificial Sequence
  GIP, GLP-1 or GLP-2 variants
  PEPTIDE (1)..(9): GIP
NDWKHNITQ

SEQ ID NO: 19 - protein, 42 aa, Mus musculus
YAEGTFISDYSIAMDKIRQQDFVNWLLAQRGKKNDWKHNITQ

SEQ ID NO: 20 - protein, 42 aa, Rattus rattus
YAEGTFISDYSIAMDKIRQQDFVNWLLAQKGKKNDWKHNITQ

SEQ ID NO: 21 - protein, 30 aa, Mus musculus
YAEGTFISDYSIAMDKIRQQDFVNWLLAQR

SEQ ID NO: 22 - protein, 30 aa, Rattus rattus
YAEGTFISDYSIAMDKIRQQDFVNWLLAQK

SEQ ID NO: 23 - protein, 33 aa, Artificial Sequence
  GIP, GLP-1 or GLP-2 variants
  PEPTIDE (1)..(33): GLP-2
HADGSFSDEMNTILDNKAARDFINWLIQTRITD

SEQ ID NO: 24 - protein, 39 aa, Homo sapiens
HGEGTFTSDLSKQMEEEAVRLFIEWLKNGGPSSGAPPPS

SEQ ID NO: 25 - protein, 30 aa, Artificial Sequence
  GIP, GLP-1 or GLP-2 variants
  PEPTIDE (1)..(30): Ex-4
HGEGTFTSDLSKQMEEEAVRLFIEWLKNGG

SEQ ID NO: 26 - protein, 31 aa, Artificial Sequence
  GIP, GLP-1 or GLP-2 variants
  PEPTIDE (1)..(31): Ex-4
DLSKQMEEEAVRLFIEWLKNGGPSSGAPPPS

SEQ ID NO: 27 - protein, 22 aa, Artificial Sequence
  GIP, GLP-1 or GLP-2 variants
  PEPTIDE (1)..(22): Ex-4
DLSKQMEEEAVRLFIEWLKNGG

SEQ ID NO: 28 - protein, 30 aa, Artificial Sequence
  GIP, GLP-1 or GLP-2 variants
  PEPTIDE (1)..(30): GIP
YPEGTFISDYSIAMDKIHQQDFVNWLLAQK

SEQ ID NO: 29 - protein, 30 aa, Artificial Sequence; identical to SEQ ID NO: 4
  GIP, GLP-1 or GLP-2 variants
  PEPTIDE (3)..(3): gamma-(CH2NH)-Glu3 (gamma-methyl-glutamate)

SEQ ID NO: 30 - protein, 30 aa, Artificial Sequence
  GIP, GLP-1 or GLP-2 variants
  PEPTIDE (2)..(2): P-Serine (phopsho-Serine)
YSEGTFISDYSIAMDKIHQQDFVNWLLAQK

SEQ ID NO: 31 - protein, 30 aa, Artificial Sequence
  GIP, GLP-1 or GLP-2 variants
  PEPTIDE (1)..(30): GIP
YVEGTFISDYSIAMDKIHQQDFVNWLLAQK

SEQ ID NO: 32 - protein, 30 aa, Artificial Sequence
  GIP, GLP-1 or GLP-2 variants
  PEPTIDE (1)..(30): GIP
YGEGTFISDYSIAMDKIHQQDFVNWLLAQK

SEQ ID NO: 33 - protein, 30 aa, Artificial Sequence; identical to SEQ ID NO: 30
  GIP, GLP-1 or GLP-2 variants
  PEPTIDE (1)..(30): GIP

SEQ ID NO: 34 - protein, 30 aa, Artificial Sequence; identical to SEQ ID NO: 4
  GIP, GLP-1 or GLP-2 variants
  PEPTIDE (1)..(1): D-Tyrosine

SEQ ID NO: 35 - protein, 30 aa, Artificial Sequence; identical to SEQ ID NO: 4
  GIP, GLP-1 or GLP-2 variants
  PEPTIDE (3)..(3): D-Glutamate

(19) United States
(12) Patent Application Publication
(10) Pub. No.: US 2020/0261543 A1
(43) Pub. Date: Aug. 20, 2020

(54) COMBINATION THERAPY FOR TREATMENT OF BONE DISORDERS

(71) Applicant: University of Copenhagen, Copenhagen K (DK)

(72) Inventors: Bolette Hartmann, Hellerup (DK); Maria Buur Nordskov Gabe, Værløse (DK); Lærke Smidt Gasbjerg, Vanløse (DK); Mette Marie Rosenkilde, Hellerup (DK); Jens Juul Holst, Hellerup (DK); Kirsa Skov-Jeppesen, Copenhagen SV (DK)

(21) Appl. No.: 16/754,944

(22) PCT Filed: Oct. 11, 2018
(86) PCT No.: PCT/EP2018/077724

§ 371 Date: Apr. 9, 2020

(30) Foreign Application Priority Data
Oct. 12, 2017 (EP) 17196171.7
Apr. 18, 2018 (EP) 18167870.7

(51) Int. Cl. A61K 38/26; A61P 19/08

(52) U.S. Cl. CPC A61K 38/26; A61K 45/06; A61P 19/08

(57) ABSTRACT

Provided herewith is the use of glucose-dependent insulinotropic peptide (GIP) and glucagon-like peptide-2 (GLP-2) for treatment of bone disorders such as osteoporosis.

### TECHNICAL FIELD

The present invention relates to the use of glucose-dependent insulinotropic peptide (GIP) and glucagon-like peptide-2 (GLP-2) for treatment of bone disorders such as osteoporosis.

### BACKGROUND

Gastrointestinal peptides and adipokines are critical signalling molecules involved in controlling whole-body energy homeostasis. These circulating hormones regulate a variety of biological responses such as hunger, satiety and glucose uptake. In vivo experiments have established that these hormones also regulate bone metabolism, while associations between these hormones and bone mass have been observed in human clinical studies.

Incretins are gastrointestinal hormones that help to regulate carbohydrate metabolism in response to food intake. The two main incretins are glucose-dependent insulinotropic peptide (GIP) and glucagon-like peptide-1 (GLP-1), both secreted by intestinal epithelial cells. Intestinal glucagon-like peptide-2 (GLP-2) is co-secreted along with GLP-1 upon nutrient ingestion.

Gastrointestinal hormones released after meal ingestion, such as GIP, GLP-1 and GLP-2 have been shown to regulate bone turnover; GIP has a positive effect on bone, and GLP-1 and GLP-2 regulate bone homeostasis and have a positive contribution to bone mass. However, their effects are often short-lived; therefore, other pharmacological interventions such as GLP-1R agonists and DPP-4 inhibitors in conjunction with GLP-2 injection are emerging as better candidates for preventing bone resorption.

Osteoporosis can be defined as a combination of reduced bone mass and altered bone quality, resulting in decreased bone strength with an increased risk of fractures. Gastrointestinal hormones including glucose-dependent insulinotropic peptide (GIP), glucagon-like peptide-1 (GLP-1) and glucagon-like peptide-1 (GLP-2) have each been implicated in bone metabolism and as potential therapies for treating osteoporosis. GLP-2 and GLP-1 are suggested for treating osteoporosis, alone or in combination with anti-osteoporosis compounds (WO 2002/024214). A dual agonist of GIP and GLP-1 is disclosed in WO2012167744. A dual agonist of the glucagon receptor and for example GIP or GLP-2 is disclosed in WO2012138941. WO2015038938 refers to a GLP-1 R and GIPR dual agonist. Room for improvement remains in the potential therapy of bone disorders associated with reduced bone density, such as osteoporosis.

### SUMMARY

The present inventors have surprisingly found that co-administration of GIP and GLP-2 induces a pronounced reduction in bone resorption and together have a synergistic effect on reduction in bone resorption. This provides a new treatment option for treatment of bone disorders.

It is an aspect to provide a composition comprising, separately or together, a GIPR agonist and a GLP-2R agonist, such as a GIP peptide and a GLP-2 peptide, for use in a method of inhibiting bone resorption and/or stimulating bone formation.

It is also an aspect to provide a composition comprising, separately or together, a GIPR agonist and a GLP-2R agonist, such as a GIP peptide and a GLP-2 peptide, for use in a method of treating a bone disorder.

In one embodiment said composition further comprises, separately or together, a GLP-1R agonist, such as a GLP-1 peptide, for use in a method of treating a bone disorder.

In one embodiment said bone disorder is selected from the group consisting of osteopenia, osteoporosis, severe osteoporosis, osteomalacia, rickets, osteitis fibrosa cystica (OFC) and Paget's disease of bone.

### DESCRIPTION OF DRAWINGS

FIG. 1: Measurements of serum C-terminal cross-linking telopeptide of type I collagen (CTX) in blood samples collected at 30 minutes interval after injection of hGLP-2 alone, hGIP alone or placebo (cf. Example 1).

FIG. 2: Measurements of CTX in blood samples collected at 30 minutes interval after injection of hGLP-2 and hGIP, or placebo (cf. Example 2).

FIG. 3: Measurements of CTX in blood samples collected after injection of hGLP-2 alone, hGIP alone or placebo (cf. Example 3).

FIG. 4: Measurements of serum N-terminal propeptide of type 1 collagen (P1NP) in blood samples collected after injection of hGLP-2 alone, hGIP alone or placebo (cf. Example 3).

FIG. 5: Measurements of serum parathyroid hormone (PTH) in blood samples collected after injection of hGLP-2 alone, hGIP alone or placebo (cf. Example 3).

FIG. 6: Measurements of CTX in blood samples collected after injection of hGLP-2 and hGIP alone or in combination, or placebo (cf. Example 4).

FIG. 7: Measurements of P1NP in blood samples collected after injection of hGLP-2 and hGIP alone or in combination, or placebo (cf. Example 4).

FIG. 8: Measurements of PTH in blood samples collected after injection of hGLP-2 and hGIP alone or in combination, or placebo (cf. Example 4).

FIG. 9: Measurements of CTX in blood samples collected after injection of hGLP-1 alone, or placebo (cf. Example 5).

FIG. 10: Nocturnal CTX: Measurements of CTX in blood samples collected after injection of hGLP-2 and hGIP alone or in combination, or placebo, administered at night-time (10 p.m.) (cf. Example 6).

### DEFINITIONS

The term “affinity” refers to the strength of binding between a receptor and its ligand(s).

The term “agonist” in the present context refers to a peptide as defined herein, capable of binding to and activating a receptor. Full agonists bind to and activate a receptor with the maximum response that an agonist can elicit at the receptor. Partial agonists also bind and activate a given receptor, but have partial efficacy at the receptor relative to a full agonist, even at maximal receptor occupancy. A selective agonist is selective for a specific type of receptor.

As used herein a GIP peptide is a peptide derived from or related to native hGIP (SEQ ID NO:1) which is a GIPR agonist. As used herein a GLP-2 peptide is a peptide derived from or related to native hGLP-2 (SEQ ID NO:2) which is a GLP2R agonist. As used herein a GLP-1 peptide is a peptide derived from or related to native hGLP-1 (SEQ ID NO:7,8,9) which is a GLP1R agonist.

An “amino acid residue” can be a natural or non-natural amino acid residue linked by peptide bonds or bonds different from peptide bonds. The amino acid residues can be in D-configuration or L-configuration. An amino acid residue comprises an amino terminal part (NH₂) and a carboxy terminal part (COOH) separated by a central part comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822(b)(2) belong to the group of amino acids listed herewith: Y,G,F,M,A,S,I,L,T,V,P,K,H,Q,E,W,R,D,N and C. Non-natural amino acids are those not listed immediately above. Also, non-natural amino acid residues include, but are not limited to, modified amino acid residues, L-amino acid residues, and stereoisomers of D-amino acid residues.

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”.

Within the meaning of the term “equivalent amino acid substitution” as applied herein, one amino acid may be substituted for another, in one embodiment, within the groups of amino acids indicated herein below:

Amino acids having polar side chains (Asp, Glu, Lys, Arg, His, Asn, Gin, Ser, Thr, Tyr, and Cys); Amino acids having non-polar side chains (Gly, Ala, Val, Leu, lie, Phe, Trp, Pro, and Met); Amino acids having aliphatic side chains (Gly, Ala Val, Leu, lie); Amino acids having cyclic side chains (Phe, Tyr, Trp, His, Pro); Amino acids having aromatic side chains (Phe, Tyr, Trp); Amino acids having acidic side chains (Asp, Glu); Amino acids having basic side chains (Lys, Arg, His); Amino acids having amide side chains (Asn, Gin); Amino acids having hydroxy side chains (Ser, Thr); Amino acids having sulphur-containing side chains (Cys, Met); Neutral, weakly hydrophobic amino acids (Pro, Ala, Gly, Ser, Thr); Hydrophilic, acidic amino acids (Gln, Asn, Glu, Asp); and Hydrophobic amino acids (Leu, lie, Val).

Where the L or D form (optical isomers) has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

A “functional variant” of a peptide is a peptide capable of performing essentially the same functions as the peptide it is a functional variant of. In particular, a functional variant can bind the same molecules, preferably with the same affinity, as the peptide it is a functional variant of.

A “bioactive agent” (i.e. a biologically active substance/agent) is any agent, drug, compound, composition of matter or mixture which provides some pharmacologic, often beneficial, effect that can be demonstrated in vivo or in vitro. It refers to the peptide sequences defined herewith, compounds or compositions comprising these and nucleic acid constructs encoding said peptides. As used herein, this term further includes any physiologically or pharmacologically active substance that produces a localized or systemic effect in an individual. A ‘bioactive agent’ as used herein denotes collectively a peptide, a nucleic acid construct encoding said peptide, and a composition comprising a peptide.

The terms “drug” and “medicament” as used herein include biologically, physiologically, or pharmacologically active substances that act locally or systemically in the human or animal body.

The terms “treatment” and “treating” as used herein refer to the management and care of a patient for the purpose of combating a condition, disease or disorder. The term is intended to include the full spectrum of treatments for a given condition from which the patient is suffering, and refer equally to curative therapy, prophylactic or preventative therapy and ameliorating or palliative therapy, such as administration of the peptide or composition for the purpose of: alleviating or relieving symptoms or complications; delaying the progression of the condition, partially arresting the clinical manifestations, disease or disorder; curing or eliminating the condition, disease or disorder; amelioration or palliation of the condition or symptoms, and remission (whether partial or total), whether detectable or undetectable; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The term “palliation”, and variations thereof, as used herein, means that the extent and/or undesirable manifestations of a physiological condition or symptom are lessened and/or time course of the progression is slowed or lengthened, as compared to not administering compositions of the present invention.

The term “Individual” refers to vertebrates, particular members of the mammalian species, preferably primates including humans. As used herein, ‘subject’ and ‘individual’ may be used interchangeably. Treatment of animals, such as mice, rats, dogs, cats, cows, horses, sheep and pigs, is, however, also within the scope of the present invention.

An “individual in need thereof” refers to an individual who may benefit from treatment. In one embodiment, said individual in need thereof is a diseased individual, wherein said disease may be a bone disorder.

A “treatment effect” or “therapeutic effect” is manifested if there is a change in the condition being treated, as measured by the criteria constituting the definition of the terms “treating” and “treatment.” There is a “change” in the condition being treated if there is at least 5% improvement, preferably 10% improvement, more preferably at least 25%, even more preferably at least 50%, such as at least 75%, and most preferably at least 100% improvement. The change can be based on improvements in the severity of the treated condition in an individual, or on a difference in the frequency of improved conditions in populations of individuals with and without treatment with the bioactive agent, or with the bioactive agent in combination with a pharmaceutical composition of the present invention.

A treatment according to the invention can be prophylactic, ameliorating and/or curative.

“Pharmacologically effective amount”, “pharmaceutically effective amount” or “physiologically effective amount” of a “bioactive agent” is the amount of a bioactive agent present in a pharmaceutical composition as described herein that is needed to provide a desired level of active agent in the bloodstream or at the site of action in an individual (e.g. the lungs, the gastric system, the colorectal system, prostate, etc.) to be treated to give an anticipated physiological response when such composition is administered.

“Co-administering” or “co-administration” as used herein refers to the administration of two or more bioactive agents. The at least two components can be administered separately, sequentially or simultaneously.

### DETAILED DESCRIPTION

GIP refers to glucose-dependent insulinotropic polypeptide, also known as Gastric Inhibitory Peptide (or polypeptide). As used herein the abbreviation hGIP is human GIP (Uniprot accession number P09681). GIP is derived from a 153-amino acid proprotein and circulates as a biologically active 42-amino acid peptide (positions 52-93). It is synthesized by K cells of the mucosa of the duodenum and the jejunum of the gastrointestinal tract.

Under physiological conditions the 42 amino acid hormone, GIP, is degraded by the enzyme dipeptidylpeptidase 4 (DPP-4), which cleaves at the third position of the GIP molecule to yield GIP3-42. GIP1-30 is produced as a result of post-translational processing. If GIP1-30 is secreted into the circulation in humans, the cleavage catalyzed by DPP-4 would result in GIP3-30.

The sequence of native hGIP is:

(SEQ ID NO: 1)
YAEGTFISDYSIAMDKIHQQDFVNWLLAQKGKKNDWKHNITQ.

GIPR (or GIP receptor) refers to gastric inhibitory polypeptide receptor(s). These seven-transmembrane proteins are found at least on beta-cells in the pancreas. As used herein the abbreviation hGIPR is human GIPR (Uniprot accession number P48546).

Several physiological effects of GIP have been identified. GIP induces insulin secretion stimulated primarily by hyperosmolarity of glucose in the duodenum. The amount of insulin secreted is greater when glucose is administered orally than intravenously. GIP is also thought to have significant effects on fatty acid metabolism through stimulation of lipoprotein lipase activity in adipocytes. GIP recently appeared as a major player in bone remodelling, and deficiency in GIP receptors has been associated with a dramatic decrease in bone quality and a subsequent increase in fracture risk.

Glucagon-like peptide-2 (GLP-2) is a 33 amino acid peptide in humans created by specific post-translational proteolytic cleavage of proglucagon in a process that also liberates the related glucagon-like peptide-1 (GLP-1) and glucagon itself. GLP-2 is produced by the intestinal endocrine L cell and by various neurons in the central nervous system. Intestinal GLP-2 is co-secreted along with GLP-1 upon nutrient ingestion. When externally administered, GLP-2 produces a number of effects in humans and rodents, including intestinal growth, enhancement of intestinal function, reduction in bone breakdown and neuroprotection. GLP-2 and related analogs have potential as treatments for short bowel syndrome, Crohn's disease, osteoporosis and as adjuvant therapy during cancer chemotherapy.

The sequence of native hGLP-2 is:

(SEQ ID NO: 2)
HADGSFSDEMNTILDNLAARDFINWLIQTKITD

The GLP-2 receptor (GLP2R) and the GLP-1 receptor (GLP1R) are G protein-coupled receptor superfamily members. GLP2R is expressed in the gut and closely related to the glucagon receptor (GCGR) and the receptor for GLP1 (GLP1R). GLP1R is expressed on beta cells of the pancreas. As used herein the abbreviation hGLP2R is human GLP2R (e.g. Uniprot accession number 095838). As used herein the abbreviation hGLP1R is human GLP1R (GLP-1 receptor) (e.g. Uniprot accession number P43220).

Glucagon-like peptide-1 (GLP-1) derives from the tissue-specific posttranslational processing of proglucagon. It is produced and secreted by intestinal enteroendocrine L-cells and certain neurons within the nucleus of the solitary tract in the brainstem upon food consumption. In the intestinal L cells proglucagon is processed to C-terminally amidated GLP-1 (7-36) and small amounts C-terminally glycine-extended GLP-1 (7-37) and released in response to meal/glucose ingestion. Active GLP-1 composes two α-helices from amino acid position 13-20 and 24-35 separated by a linker region.

GLP-1 decreases blood sugar levels in a glucose-dependent manner by enhancing the secretion of insulin. Beside the insulinotropic effects, GLP-1 has been associated with numerous regulatory and protective effects. The action of GLP-1 is preserved in patients with type 2 diabetes and substantial pharmaceutical research has therefore been directed towards the development of GLP-1-based treatment. Endogenous GLP-1 is rapidly degraded primarily by dipeptidyl peptidase-4 (DPP-4), but also neutral endopeptidase 24.11 (NEP 24.11) and renal clearance, resulting in a half-life of approximately 2 minutes. Consequently, only 10-15% of GLP-1 reaches circulation intact, leading to fasting plasma levels of only 0-15 pmol/L. To overcome this, combination with DPP-4 inhibitors and the development degradation resistant variants of GLP-1 have been employed.

The sequences of native hGLP-1 are:

(SEQ ID NO: 7)
hGLP-1(1-37): HDEFERHAEGTFTSDVSSYLEGQAAKEFIAWLVKGR
G
(SEQ ID NO: 8)
hGLP-1(7-36): HAEGTFTSDVSSYLEGQAAKEFIAWLVKGR
(SEQ ID NO: 9)
hGLP-1(7-37): HAEGTFTSDVSSYLEGQAAKEFIAWLVKGRG

### Treatment of Bone Disorders

It is an aspect of the present disclosure to provide a composition comprising, separately or together, a GIPR agonist and a GLP-2R agonist, for use in a method of inhibiting bone resorption and/or stimulating bone formation.

It is also aspect of the present disclosure to provide a composition comprising, separately or together, a GIP peptide and a GLP-2 peptide, for use in a method of inhibiting bone resorption and/or stimulating bone formation.

Also provided is the use of a composition comprising, separately or together, a GIP peptide and a GLP-2 peptide, for the manufacture of a medicament for inhibiting bone resorption and/or stimulating bone formation.

Also provided is a method of inhibiting bone resorption and/or stimulating bone formation, said method comprising administering a therapeutically effective amount of a composition comprising, separately or together, a GIP peptide and a GLP-2 peptide, to an individual in need thereof.

Also disclosed is a composition comprising, separately or together, a GIPR agonist, a GLP-2R agonist and a GLP-1R agonist; such as a GIP peptide, a GLP-2 peptide and a GLP-1 peptid; for use in a method of inhibiting bone resorption and/or stimulating bone formation.

In one embodiment there is provided a composition comprising, separately or together, a GIPR agonist and a GLP-2R agonist, for use in a method of treating a bone disorder.

In one embodiment there is provided a composition comprising, separately or together, a GIP peptide and a GLP-2 peptide, for use in a method of treating a bone disorder.

Also provided is the use of a composition comprising, separately or together, a GIP peptide and a GLP-2 peptide, for the manufacture of a medicament for use in a method of treating a bone disorder.

Also provided is a method of treating a bone disorder, said method comprising administering a therapeutically effective amount of a composition comprising, separately or together, a GIP peptide and a GLP-2 peptide, to an individual in need thereof.

Also disclosed is a composition comprising, separately or together, a GIPR agonist, a GLP-2R agonist and a GLP-1R agonist; such as a GIP peptide, a GLP-2 peptide and a GLP-1 peptide; for use in a method of treating a bone disorder.

In one embodiment the bone disorder is a disorder associated with increased bone resorption and/or reduced bone formation.

In one embodiment the bone disorder is associated with poor or reduced bone density.

A method of treating a bone disorder as used herein may include one or more of treating, preventing and alleviating said bone disorder.

Bone density or bone mineral density (BMD) is the amount of bone mineral in bone tissue. The concept is of mass of mineral per volume of bone (relating to density in the physics sense), although clinically it is measured by proxy according to optical density per square centimeter of bone surface upon imaging. Bone density measurement is used in clinical medicine as an indirect indicator of osteoporosis/osteopenia and fracture risk. It is measured by a procedure called densitometry. There is a statistical association between poor bone density and higher probability of fracture. Bone density measurements are used to screen people for osteoporosis risk and to identify those who might benefit from measures to improve bone strength.

The T-score is the relevant measure when screening for osteoporosis. It is the bone mineral density (BMD) at the site when compared to the young normal reference mean. The criteria of the World Health Organization are:

Normal is a T-score of −1.0 or higher
Osteopenia is defined as between −1.0 and −2.5
Osteoporosis is defined as −2.5 or lower, meaning a bone density that is two and a half standard deviations below the mean of a young normal reference.

In one embodiment the bone disorder is associated with a T-score of −1.0 or lower, such as between −1.0 and −2.5, such as −2.5 or lower.

An individual in need as referred to herein, may in one embodiment be an individual that may benefit from the administration of a composition comprising, separately or together, a GIPR agonist and a GLP-2R agonist, such as a GIP peptide and a GLP-2 peptide.

Such an individual may suffer from a bone disorder or be in risk of suffering therefrom. The individual may be any human being, male or female, infant, middle-aged or old. The disorder to be treated or prevented in the individual may relate to the age of the individual, the general health of the individual, the medications used for treating the individual and whether or not the individual has a prior history of suffering from diseases or disorders that may have or have induced a bone density disorder.

In one embodiment the bone disorder is selected from the group consisting of osteopenia, osteoporosis, severe osteoporosis, osteomalacia, rickets, osteitis fibrosa cystica (OFC) and Paget's disease of bone. In one embodiment the bone disorder is osteopenia. In one embodiment the bone disorder is osteoporosis.

It follows that in one embodiment there is provided a composition comprising, separately or together, a GIPR agonist and a GLP-2R agonist, such as a GIP peptide and a GLP-2 peptide, for use in a method of treating a bone disorder selected from the group consisting of osteopenia, osteoporosis, severe osteoporosis, osteomalacia, rickets, osteitis fibrosa cystica (OFC) and Paget's disease of bone.

Also provided is the use of a composition comprising, separately or together, a GIPR agonist and a GLP-2R agonist, such as a GIP peptide and a GLP-2 peptide, for the manufacture of a medicament for use in a method of treating a bone disorder selected from the group consisting of osteopenia, osteoporosis, severe osteoporosis, osteomalacia, rickets, osteitis fibrosa cystica (OFC) and Paget's disease of bone.

Also provided is a method of treating a bone disorder selected from the group consisting of osteopenia, osteoporosis, severe osteoporosis, osteomalacia, rickets, osteitis fibrosa cystica (OFC) and Paget's disease of bone, said method comprising administering a therapeutically effective amount of a composition comprising, separately or together, a GIPR agonist and a GLP-2R agonist, such as a GIP peptide and a GLP-2 peptide, to an individual in need thereof.

In one embodiment there is provided is a composition comprising, separately or together, a GIPR agonist, a GLP-2R agonist and a GLP-1R agonist; such as a GIP peptide, a GLP-2 peptide and a GLP-1 peptide; for use in a method of treating a bone disorder selected from the group consisting of osteopenia, osteoporosis, severe osteoporosis, osteomalacia, rickets, osteitis fibrosa cystica (OFC) and Paget's disease of bone.

In one embodiment there is provided a composition comprising, separately or together, a GIPR agonist and a GLP-2R agonist, such as a GIP peptide and a GLP-2 peptide, for use in a method of treating a bone disorder selected from the group consisting of osteopenia, osteoporosis and severe osteoporosis.

In one embodiment there is provided a composition comprising, separately or together, a GIPR agonist and a GLP-2R agonist, such as a GIP peptide and a GLP-2 peptide, for use in a method of treating osteopenia.

In one embodiment there is provided a composition comprising, separately or together, a GIPR agonist and a GLP-2R agonist, such as a GIP peptide and a GLP-2 peptide, for use in a method of treating osteoporosis.

### GIP, GLP-2 and GLP-1 Peptides

A composition comprising, separately or together, a GIP peptide and a GLP-2 peptide as defined herein comprises at least two active pharmaceutical ingredients; a GIP peptide and a GLP-2 peptide.

In some embodiments the composition further comprises a GLP-1 peptide.

As used herein a GIP peptide in one embodiment refers to a peptide having a sequence SEQ ID NO:1, or a functional variant or functional fragment thereof; a GLP-2 peptide in one embodiment refers to a peptide having a sequence SEQ ID NO:2, or a functional variant or functional fragment thereof; and a GLP-1 peptide in one embodiment refers to a peptide having a sequence SEQ ID NO:7, :8 or :9, or a functional variant or functional fragment thereof.

In one embodiment a functional variant of a GIP peptide, such as SEQ ID NO:1, a functional variant of a GLP-2 peptide, such as SEQ ID NO:2, and a functional variant of a GLP-1 peptide, such as SEQ ID NO:7, :8 or :9, has at least 60% sequence identity, such as at least 70% sequence identity, such as at least 75% sequence identity, such as at least 80% sequence identity, such as at least 85% sequence identity, such as at least 90% sequence identity, such as at least 95% sequence identity, such as at least 97% sequence identity to said GIP peptide, such as SEQ ID NO:1, said GLP-2 peptide, such as SEQ ID NO:2 and/or said GLP-1 peptide.

In one embodiment a functional variant of a GIP peptide, such as SEQ ID NO:1, a functional variant of a GLP-2 peptide, such as SEQ ID NO:2, and/or a functional variant of a GLP-1 peptide, such as SEQ ID NO:7, :8 or :9, has 60 to 65% sequence identity, such as 65 to 70% sequence identity, such as 70 to 75% sequence identity, such as 75 to 80% sequence identity, such as 80 to 85% sequence identity, such as 85 to 90% sequence identity, such as 90 to 95% sequence identity, such as 95 to 99% sequence identity, such as 99 to 100% sequence identity to said peptide. ‘Identity’ and ‘sequence identity’ may be used interchangeably herein.

In one embodiment a functional variant comprises one or more amino acid substitutions, such as 1 to 8 amino acid substitutions, such as 1 to 2, 2 to 3, 3 to 4, 4 to 5, 5 to 6, 6 to 7 or 7 to 8 amino acid substitutions.

In one embodiment a functional variant comprises one amino acid substitution, two amino acid substitutions, three amino acid substitutions, four amino acid substitutions or five amino acid substitutions.

In one embodiment said amino acid substitutions are conservative amino acid substitutions. In one embodiment said functional variant comprises one or more conservative amino acid substitutions.

A conservative substitution (or synonymous substitution) is the substitution of amino acids whose side chains have similar biochemical properties and thus do not affect the function of the peptide.

Among the common amino acids, for example, a “conservative amino acid substitution” can also be illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

In one embodiment, a serine residue of a peptide disclosed herein is substituted with an amino acid selected from the group consisting of Gin, Asn and Thr (all amino acids with polar uncharged side chains); and independently thereof, a glycine residue (Gly) is substituted with an amino acid selected from the group consisting of Ala, Val, Leu, and lie; and independently thereof, an arginine residue (Arg) is substituted with an amino acid selected from the group consisting of Lys and His (all have positively charged side chains); and independently thereof, a lysine residue (Lys) is substituted with an amino acid selected from the group consisting of Arg and His; and independently thereof, a methionine residue (Met) is substituted with an amino acid selected from the group consisting of Leu, Pro, Ile, Val, Phe, Tyr and Trp (all have hydrophobic side chains); and independently thereof, a glutamine residue (Gln) is substituted with an amino acid selected from the group consisting of Asp, Glu, and Asn; and independently thereof, an alanine residue (Ala) is substituted with an amino acid selected from the group consisting of Gly, Val, Leu, and Ile.

Particular amino acid substitutions as defined herein are K to R, E to D, L to M, Q to E, I to V, I to L, A to S, Y to W, K to Q, S to T, N to S and Q to R.

Other particular amino acid substitutions as defined herein are T to K, L to K, N to K, A to K and R to K.

The identity between amino acid sequences may be calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90, or by simple comparison of the specific amino acids present at corresponding positions in two peptide sequences to be compared. Homology may be used as a synonym to identity/sequence identity.

Conservative substitutions may be introduced in any one or more positions of a peptide according to the present disclosure, as long as the variant remains functional. It may however also be desirable to introduce non-conservative substitutions in one or more positions (non-synonymous substitutions).

A non-conservative substitution leading to the formation of a variant of the disclosed peptides in one embodiment comprises substitution of amino acid residues that i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gin or a charged amino acid such as Asp, Glu, Arg, or Lys, or substituting a charged or a polar residue for a non-polar one; and/or ii) differ substantially in its effect on peptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids can in one embodiment be made based upon their hydrophobicity and hydrophilicity values and the relative similarity of the amino acid side-chain substituents, including charge, size, and the like.

The peptides as disclosed herein in one embodiment comprise proteinogenic or natural amino acids, i.e. the 22 amino acids naturally incorporated into polypeptides. Of these, 20 are encoded by the universal genetic code and the remaining 2; selenocysteine (Sec, U) and pyrrolysine (Pyl, O), are incorporated into proteins by unique synthetic mechanisms.

A peptide according to the present disclosure in one embodiment comprises one or more non-naturally occurring amino acid residues (unnatural, non-proteinogenic or non-standard amino acids). Non-naturally occurring amino acids include e.g., without limitation, beta-2-naphthyl-alanine, trans-3-methylproline, 2,4-methanoproline, cis-4-hydroxyproline, ornithine, trans-4-hydroxyproline, N-methylglycine, allo-threonine, methylthreonine, hydroxyethylcysteine, hydroxyethylhomocysteine, nitroglutamnine, homoglutamine, pipecolic acid, thiazolidine carboxylic acid, dehydroproline, 3- and 4-methylproline, 3,3-dimethylproline, tert-leucine, norleucine, norvaline, 2-azaphenylalanine, 3-azaphenylalanine, 4-azaphenylalanine, and 4-fluorophenylalanine.

Any amino acids as defined herein may be in the L- or D-configuration. If nothing is specified, reference to the L-isomeric form is preferably meant.

The standard and/or non-standard amino acids may be linked by peptide bonds (to form a linear peptide chain), or by non-peptide bonds (e.g. via the variable side-chains of the amino acids). Preferably, the amino acids of the present disclosure are linked by peptide bonds.

In one embodiment a functional fragment of a GIP peptide, such as SEQ ID NO:1, comprises or consists of a consecutive stretch of amino acids of SEQ ID NO:1, or a variant thereof, said consecutive stretch comprising or consisting of 41 amino acids or less of SEQ ID NO:1, or a variant thereof, such as 10-15 amino acids, such as 15-20 amino acids, such as 20-25 amino acids, such as 25-30 amino acids, such as 30-35 amino acids, such as 35-41 amino acids of SEQ ID NO:1, or a variant thereof.

In one embodiment a functional fragment of GLP-2 peptide, such as SEQ ID NO:2, comprises or consists of a consecutive stretch of amino acids of SEQ ID NO:2, or a variant thereof, said consecutive stretch comprising or consisting of 32 amino acids or less of SEQ ID NO:2, or a variant thereof, such as 10-15 amino acids, such as 15-20 amino acids, such as 20-25 amino acids, such as 25-30 amino acids, such as 30-32 amino acids of SEQ ID NO:2, or a variant thereof.

In one embodiment a functional fragment of GLP-1 peptide, such as SEQ ID NO:7, :8 or :9, comprises or consists of a consecutive stretch of amino acids of SEQ ID NO:7, :8 or :9, or a variant thereof, said consecutive stretch comprising or consisting of 37 amino acids or less of SEQ ID NO:7, or a variant thereof, such as 10-15 amino acids, such as 15-20 amino acids, such as 20-25 amino acids, such as 25-30 amino acids, such as 30-31 amino acids of SEQ ID NO:7, 8: or 9:, or a variant thereof.

The terms ‘peptide’ and ‘isolated peptide’ may be used interchangeably herein. The terms ‘variant’ and ‘functional variant’ may be used interchangeably herein. The terms ‘fragment’ and ‘functional fragment’ may be used interchangeably herein.

In one embodiment the peptide is non-naturally occurring.

In one embodiment the peptide is synthetic.

In one embodiment the peptide is an isolated peptide.

In another embodiment, a variant as defined herein includes sequences wherein an alkyl amino acid is substituted for an alkyl amino acid, wherein an aromatic amino acid is substituted for an aromatic amino acid, wherein a sulfur-containing amino acid is substituted for a sulfur-containing amino acid, wherein a hydroxy-containing amino acid is substituted for a hydroxy-containing amino acid, wherein an acidic amino acid is substituted for an acidic amino acid, wherein a basic amino acid is substituted for a basic amino acid, and/or wherein a dibasic monocarboxylic amino acid is substituted for a dibasic monocarboxylic amino acid.

The term peptide also embraces post-translational modifications introduced by chemical or enzyme-catalyzed reactions, as are known in the art. These include acetylation, phosphorylation, methylation, glucosylation, glycation, amidation, hydroxylation, deimination, deamidation, carbamylation and sulfation of one or more amino acid residues, and also proteolytic modification by known proteinases including lysosomal kathepsins, and also calpains, secretases and matrix-metalloproteinases.

In one embodiment the GIP peptide and/or the GLP-2 peptide and/or the GLP-1 peptide is C-terminally amidated (—NH₂).

In one embodiment the GIP peptide and/or the GLP-2 peptide and/or the GLP-1 peptide is N-terminally acetylated (COCH₃).

Also, functional equivalents of the peptides may comprise chemical modifications such as ubiquitination, labeling (e.g., with radionuclides, various enzymes, etc.), pegylation (derivatization with polyethylene glycol), or by insertion (or substitution by chemical synthesis) of amino acids such as ornithine, which do not normally occur in human proteins (non-proteinogenic).

Sterically similar compounds may be formulated to mimic the key portions of the peptide structure. This may be achieved by techniques of modelling and chemical designing known to those of skill in the art. For example, esterification and other alkylations may be employed to modify the amino terminus of e.g. a di-arginine peptide backbone, to mimic a tetra peptide structure. It will be understood that all such sterically similar constructs fall within the scope of the present invention. Peptides with N-terminal and C-terminal alkylations and esterifications are also encompassed within the present invention.

A contiguous or consecutive peptide sequence is a sequence of consecutive amino acids being linked linearly by peptide bonds. Contiguous and consecutive amino acid sequence is used interchangeably herein.

A functional variant and functional fragment as used herein means that the variant or fragment of the peptide retain all or some of the functions associated with the said peptide dual agonist, i.e. they retain at least some effect associated with the native sequence.

In one embodiment a functional variant or fragment retains the same biological activity or capabilities as the native peptide or the peptide from which it is derived.

In one embodiment a functional variant or a functional fragment of GIP, such as of SEQ ID NO:1, is capable of one or more of:
a. binding to GIPR, and/or
b. activation of GIPR, and/or
c. stimulation of GIPR-activation, such as GIPR-mediated cAMP production, and/or
d. inhibiting bone resorption, and/or
e. stimulating bone formation.

In one embodiment a functional variant or a functional fragment of GLP-2, such as of SEQ ID NO:2, is capable of one or more of:
a. binding to GLP2R, and/or
b. activation of GLP2R, and/or
c. stimulation of GLP2R-activation, such as GLP2R-mediated cAMP production, and/or
d. inhibiting bone resorption, and/or
e. stimulating bone formation.

In one embodiment a functional variant or a functional fragment of GLP-1, such as of SEQ ID NO:7, 8: or 9, is capable of one or more of:
a. binding to GLP1R, and/or
b. activation of GLP1R, and/or
c. stimulation of GLP1R-activation, such as GLP1R-mediated cAMP production, and/or
d. inhibiting bone resorption, and/or
e. stimulating bone formation.

In one embodiment a functional fragment of GIP, such as of SEQ ID NO:1, and a functional variant or a functional fragment of GLP-2, such as of SEQ ID NO:2, is a full agonist of GIPR and GLP2R, respectively.

In one embodiment said GIP peptide is a full agonist of GIPR; and/or wherein said GLP-2 peptide is a full agonist of GLP2R.

In one embodiment a GIP peptide according to the present disclosure is an analogue of hGIP; such as a protease-resistant analogue of hGIP.

In one embodiment a GLP-2 peptide according to the present disclosure is an analogue of hGLP-2; such as a protease-resistant analogue of hGLP-2.

In one embodiment a GLP-1 peptide according to the present disclosure is an analogue of hGLP-1; such as a protease-resistant analogue of hGLP-1.

In one embodiment a GIP peptide is a functional fragment of the full-length 42-amino acid hGIP peptide (GIP 1-42), which retains its GIPR agonistic properties. In one embodiment a GIP peptide is a functional variant of the 42-amino acid hGIP peptide, which retains its GIPR agonistic properties.

In one embodiment the GIP peptide of the present disclosure is resistant to DPP-4 degradation.

In one embodiment the GIP peptide is selected from the group consisting of:

(SEQ ID NO: 4)
YAEGTFISDYSIAMDKIHQQDFVNWLLAQK hGIP(1-30);
(SEQ ID NO: 14)
AEGTFISDYSIAMDKIHQQDFVNWLLAQK hGIP(2-30);
(SEQ ID NO: 15)
Y(D-Ala)EGTFISDYSIAMDKIHQQDFVNWLLAQKGKKNDWKHNITQ
D-Ala2-hGIP (SEQ ID NO: 1 with Ala at position 2
substituted with D-Ala);
(SEQ ID NO: 16)
YAPGTFISDYSIAMDKIHQQDFVNWLLAQKGKKNDWKHNITQ Pro3-
hGIP (SEQ ID NO: 1 with Glu at position 3
substituted with Pro);
(SEQ ID NO: 17)
Y(D-Ala)EGTFISDYSIAMDKIHQQDFVNWLLAQK D-Ala2-hGIP
(1-30);
(SEQ ID NO: 18)
NDWKHNITQ hGIP(34-42);
(SEQ ID NO: 28)
Pro2-GIP(1-30): YPEGTFISDYSIAMDKIHQQDFVNWLLAQK;
(SEQ ID NO: 29)
y(CH2NH)-Glu3-GIP(1-30): YAEGTFISDYSIAMDKIHQQDFVNW
LLAQK;
(SEQ ID NO: 30)
(P)Ser2-GIP(1-30): Y(P)SEGTFISDYSIAMDKIHQQDFVNWLLA
QK;
(SEQ ID NO: 31)
Val2-GIP(1-30): YVEGTFISDYSIAMDKIHQQDFVNWLLAQK;
(SEQ ID NO: 32)
Gly2-GIP(1-30): YGEGTFISDYSIAMDKIHQQDFVNWLLAQK;
(SEQ ID NO: 33)
Ser2-GIP(1-30): YSEGTFISDYSIAMDKIHQQDFVNWLLAQK;
(SEQ ID NO: 34)
D-Tyr1-GIP(1-30): (D)YAEGTFISDYSIAMDKIHQQDFVNWLLAQ
K;
(SEQ ID NO: 35)
D-Glu3-GIP(1-30): YA(D)EGTFISDYSIAMDKIHQQDFVNWLLAQ
K;

each optionally with a N-terminal H and/or a C-terminal —OH or —NH₂; or a functional variant thereof.

In one embodiment the GIP peptide is the mouse GIP (mGIP; YAEGTFISDYSIAMDKIRQQDFVNWLLAQRGKKNDWKHNITQ; SEQ ID NO:19) or the rat GIP sequence (rGIP; YAEGTFISDYSIAMDKIRQQDFVNWLLAQKGKKNDWKHNITQ; SEQ ID NO:20), such as mGIP, mGIP(1-30) (YAEGTFISDYSIAMDKIRQQDFVNWLLAQR; SEQ ID NO:21), GIP and rGIP(1-30) (YAEGTFISDYSIAMDKIRQQDFVNWLLAQK; SEQ ID NO:22).

In one embodiment a GLP-2 peptide is a functional fragment of the full-length 33-amino acid hGLP-2 peptide (GLP-2 1-33), which retains its GLP-2R agonistic properties. In one embodiment a GLP-2 peptide is a functional variant of the 33-amino acid hGLP-2 peptide, which retains its GLP-2R agonistic properties.

In one embodiment the GLP-2 peptide is selected from the group consisting of teduglutide (Gattex; revestive); glepaglutide; hGLP-2 with an extra C-terminal Arg (HADGSFSDEMNTILDNLAARDFINWLIQTKITDR; SEQ ID NO:3); Human Gly2GLP-2 (HGDGSFSDEMNTILDNLAARDFINWLIQTKITD; SEQ ID NO:5); Human Gly2,Glu28GLP-2 (HGDGSFSDEMNTILDNLAARDFINWLIETKITD; SEQ ID NO:6); acylated versions of GLP-2 (aGLP-2); a GLP-2 analogue with two substitutions (Leu17 has been replaced by Lys, and Lys30 has been replaced by Arg (HADGSFSDEMNTILDNKAARDFINWLIQTRITD; SEQ ID NO:23) and the analogue is acylated with a β-alanine spacer and a C16 fatty acid at the ε-amino group of Lys17 (cf. WO 2004/035624); or a functional variant thereof.

In one embodiment a GLP-1 peptide is a functional fragment or functional variant of the full-length 37-amino acid hGLP-1 peptide (SEQ ID NO:7), a functional fragment or functional variant of the 31-amino acid hGLP-1 peptide (SEQ ID NO:8) or a functional fragment or functional variant of the 30-amino acid hGLP-1 peptide (SEQ ID NO:9) which retains its GLP-1R agonistic properties.

In one embodiment the GLP-1 peptide is selected from the group consisting of exenatide, lixisenatide, albiglutide, liraglutide, taspoglutide, dulaglutide, semaglutide, exendin-4 (Ex4; HGEGTFTSDLSKQMEEEAVRLFIEWLKNGGPSSGAPPPS; SEQ ID NO:24), Ex4(1-30) (HGEGTFTSDLSKQMEEEAVRLFIEWLKNGG; SEQ ID NO:25), Ex4(9-39) (DLSKQMEEEAVRLFIEWLKNGGPSSGAPPPS; SEQ ID NO:26), Ex(9-30) (DLSKQMEEEAVRLFIEWLKNGG; SEQ ID NO:27), SEQ ID NO:7 hGLP-1(1-37)), (SEQ ID NO:8 hGLP-1(7-36)), SEQ ID NO:9 (hGLP-1(7-37)), SEQ ID NO:10 (hGLP-1(1-36)), SEQ ID NO:11 (hGLP-1(9-36)), SEQ ID NO:12 (A7-hGLP-1(7-36)) and SEQ ID NO:13 (A10-hGLP-1(7-36)), or a functional variant thereof.

### GIPR, GLP-2R and GLP-1R Agonists

A composition comprising, separately or together, a GIPR agonist and a GLP-2R agonist as defined herein comprises at least two active pharmaceutical ingredients; a GIPR agonist and a GLP-2R agonist.

In one embodiment said composition further comprises, separately or together, a GLP-1R agonist, such as a GLP-1 peptide.

In one embodiment a GIPR agonist as disclosed herein is capable of binding to and activating GIPR. In some embodiments, the GIPR is the human GIPR (Uniprot accession number P48546).

In one embodiment a GLP-2R agonist is capable of binding to and activating GLP2R. In some embodiments, the GLP2R is the human GLP2R (Uniprot accession number O95838).

In one embodiment a GLP-1R agonist is capable of binding to and activating GLP1R. In some embodiments, the GLP1R is the human GLP1R (Uniprot accession number P43220).

### Further Active Ingredients

It is also an aspect to provide a composition comprising, separately or together, a GIPR agonist and a GLP-2R agonist, such as a GIP peptide and a GLP-2 peptide, as defined herein for use in combination with a further active pharmaceutical ingredient.

It is also an aspect to provide a composition comprising, separately or together, a GIPR agonist, a GLP-2R agonist and a GLP-1R agonist; such as a GIP peptide, a GLP-2 peptide and a GLP-1 peptide; as defined herein for use in combination with a further active pharmaceutical ingredient.

Said further active ingredient is in one embodiment useful for treating a bone disorder, such as a bone disorder associated with reduced bone density.

In one embodiment the further active pharmaceutical ingredient is selected from the group consisting of Bisphosphonates including Alendronate (Fosamax), Risedronate (Actonel, Atelvia, Benet), Ibandronate (Boniva), Zoledronic acid (Reclast, Aclasta, Zometa), Etidronic acid (Didronel), Pamidronic acid (Aredia/Pamimed), Tiludronic acid (Skelid); estrogen replacement therapy; hormone therapies; hormone-like medications including raloxifene (Evista); Calcitonin (Fortical and Miacalcin), Denosumab (Prolia); Teriparatide (Forteo); Vitamin D (alfacalcidol or calcitriol); calcium or phosphorus supplement.

In one embodiment the composition comprising, separately or together, a GIPR agonist and a GLP-2R agonist, such as a GIP peptide and a GLP-2 peptide, as defined herein is used in combination with a further active pharmaceutical ingredient, wherein said further active pharmaceutical ingredient is a DPP-4 inhibitor (Dipeptidyl Peptidase IV Inhibitor); such as Diprotin A; or a gliptin such as sitagliptin, saxagliptin, vildagliptin and alogliptin,

### Nucleic Acid Construct

In one embodiment there is provided a nucleic acid construct (or individual constructs) encoding a GIP peptide and/or a GLP-2 peptide, and/or optionally a GLP-1 peptide, as defined herein. In one embodiment said nucleic acid construct(s) will be able to continuously express said peptide(s) for a prolonged period of time. It is thus an aspect to provide one or more nucleic acid constructs encoding a GIP peptide and/or a GLP-2 peptide, and optionally a GLP-1 peptide, as defined herewith, for use in a method of inhibiting bone resorption and/or stimulating bone formation; such as for use in for method of treating a bone disorder.

By nucleic acid construct is understood a genetically engineered nucleic acid. The nucleic acid construct may be a non-replicating and linear nucleic acid, a circular expression vector or an autonomously replicating plasmid. A nucleic acid construct may comprise several elements such as, but not limited to genes or fragments of same, promoters, enhancers, terminators, poly-A tails, linkers, polylinkers, operative linkers, multiple cloning sites (MCS), markers, STOP codons, internal ribosomal entry sites (IRES) and host homologous sequences for integration or other defined elements. It is to be understood that the nucleic acid construct according to the present invention may comprise all or a subset of any combination of the above-mentioned elements.

Methods for engineering nucleic acid constructs are well known in the art (see, e.g., Molecular Cloning: A Laboratory Manual, Sambrook et al., eds., Cold Spring Harbor Laboratory, 2nd Edition, Cold Spring Harbor, N.Y., 1989). Further, nucleic acid constructs according to the present invention may be synthesized without template, and may be obtained from various commercial suppliers (e.g. Genscript Corporation).

In one embodiment, the nucleic acid constructs are naked DNA constructs comprising sequences encoding the peptides.

It is also an aspect to provide the nucleic acid construct as described herein above comprised within a delivery vehicle. A delivery vehicle is an entity whereby a nucleotide sequence or polypeptide or both can be transported from at least one media to another. Delivery vehicles are generally used for expression of the sequences encoded within the nucleic acid construct and/or for the intracellular delivery of the construct or the polypeptide encoded therein.

In one embodiment, there is provided a delivery vehicle comprising the nucleic acid construct as defined herein. A delivery vehicle may be selected from the group consisting of: RNA based vehicles, DNA based vehicles/vectors, lipid based vehicles (such as a liposome), polymer based vehicles (such as a cationic polymer DNA carrier), colloidal gold particles (coating) and virally derived DNA or RNA vehicles or vectors.

Methods of non-viral delivery include physical (carrier-free delivery) and chemical approaches (synthetic vector-based delivery).

Physical approaches, including needle injection, gene gun, jet injection, electroporation, ultrasound, and hydrodynamic delivery, employ a physical force that permeates the cell membrane and facilitates intracellular gene transfer. Said physical force may be electrical or mechanical.

Examples of chemical delivery vehicles include, but are not limited to: biodegradable polymer microspheres, lipid based formulations such as liposome carriers, cationically charged molecules such as liposomes, calcium salts or dendrimers, lipopolysaccharides, polypeptides and polysaccharides.

Another embodiment comprises a vector which herein is denoted a viral vector (i.e. not a virus) as a delivery vehicle. Viral vectors according to the present invention are made from a modified viral genome, i.e. the actual DNA or RNA forming the viral genome, and introduced in naked form. Thus, any coat structures surrounding the viral genome made from viral or non-viral proteins are not part of the viral vector.

The virus from which the viral vector is derived may be selected from the non-exhaustive group of: adenoviruses, retroviruses, lentiviruses, adeno-associated viruses, herpesviruses, vaccinia viruses, foamy viruses, cytomegaloviruses, Semliki forest virus, poxviruses, RNA virus vector and DNA virus vector. Such viral vectors are well known in the art.

In one embodiment, said viral vectors may be selected from the group consisting of adenoviruses, lentiviruses, adeno-associated viruses (AAV) and recombinant adeno-associated viruses (rAAV). In one preferred embodiment, said viral vector is a therapeutic rAAV vector such as a therapeutic rAAV vector.

An adenovirus is a group of double-stranded DNA containing viruses. Adenoviruses can be genetically modified making them replication incompetent or conditionally replication incompetent. In this form, as adenoviral constructs or adenovectors, they can be used as gene delivery vehicles for vaccination or gene therapy.

Another aspect of relates to a cell comprising the nucleic acid construct as defined herein. Such a recombinant cell can be used a tool for in vitro research, as a delivery vehicle for the nucleic acid construct or as part of a gene-therapy regime. The nucleic acid construct can be introduced into cells by techniques well known in the art which include microinjection of DNA into the nucleus of a cell, transfection, electroporation, lipofection/liposome fusion and particle bombardment. Suitable cells include autologous and non-autologous cells, and may include xenogenic cells.

### Method of Preparation

The peptides as defined herein may be prepared by any methods known in the art; such as by standard peptide-preparation techniques including solution synthesis or Merrifield-type solid phase synthesis. Some are commercially available.

In one embodiment a peptide as defined herein is synthetically made or produced. The methods for synthetic production of peptides are well known in the art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

In one embodiment a peptide as defined herein is produced synthetically, such as by the Sequence Assisted Peptide Synthesis (SAPS) method, by solution synthesis, by Solid-phase peptide synthesis (SPPS) such as Merrifield-type solid phase synthesis, by recombinant techniques (production by host cells comprising a first nucleic acid sequence encoding the peptide operably associated with a second nucleic acid capable of directing expression in said host cells) or enzymatic synthesis. These are well-known to the skilled person.

### Pharmaceutical Composition and Formulation

Whilst it is possible for the GIP peptide, the GLP-2 peptide and the optional GLP-1 peptide as defined herewith to be administered as the raw chemical (peptide), it is sometimes preferred to present them in the form of a pharmaceutical formulation. Such a pharmaceutical formulation may be referred to as a pharmaceutical composition, pharmaceutically acceptable composition or pharmaceutically safe composition.

Accordingly, also provided is a pharmaceutical formulation such as a pharmaceutically acceptable composition, comprising separately or together a GIP peptide and a GLP-2 peptide, or a nucleic acid encoding the same, or pharmaceutically acceptable salts or esters thereof, and a pharmaceutically acceptable carrier, excipient and/or diluent. The pharmaceutical formulations may be prepared by conventional techniques, e.g. as described in Remington: The Science and Practice of Pharmacy 2005, Lippincott, Williams & Wilkins.

Pharmaceutically acceptable salts of the instant peptides, where they can be prepared, are also intended to be covered. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The peptides as disclosed herein may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

A pharmaceutically acceptable salt of the peptides as disclosed herein is in one embodiment in solution with a physiologically acceptable pH, i.e. the solution comprising the peptide salt preferably has a pH acceptable for clinical use.

### Administration and Dosage

In one embodiment of the present disclosure, a composition comprising, separately or together, a GIPR agonist and a GLP-2R agonist, such as a GIP peptide and a GLP-2 peptide, and optionally a GLP-1 peptide, is administered to individuals in need of treatment in pharmaceutically effective doses or a therapeutically effective amount. The dosage requirements will vary with the particular drug composition employed, the route of administration and the particular subject being treated, which depend on the severity and the sort of the disorder as well as on the weight and general state of the subject. It will also be recognized by one skilled in the art that the optimal quantity and spacing of individual dosages will be determined by the nature and extent of the condition being treated, the form, route and site of administration, and the particular patient being treated, and that such optima can be determined by conventional techniques. It will also be appreciated by one of skill in the art that the optimal course of treatment, i.e., the number of doses of a compound given per day for a defined number of days, can be ascertained using conventional course of treatment determination tests.

In one embodiment the composition comprising, separately or together, a GIPR agonist and a GLP-2R agonist, such as a GIP peptide and a GLP-2 peptide, and optionally a GLP-1 peptide, is administered at least once daily, such as once daily, such as twice daily, such as thrice daily, such as four times daily, such as five times daily.

A dose may also be administered in intermittent intervals, or intervals, whereby a dose is not administered every day. Rather one or more doses may be administered every second day, every third day, every fourth day, every fifth day, every sixth day, every week, every second week, every third week, every fourth week, every fifth week, every sixth week, or intervals within those ranges (such as every 2 to 4 weeks, or 4 to 6 weeks).

The skilled person knows that if the number of daily administrations is increased, the dose to be administered in each administration may be decreased accordingly.

Likewise, if the duration of each administration is decreased, the dosage may be increased accordingly.

In one embodiment said GIP peptide and said GLP-2 peptide are administered simultaneously, sequentially or separately.

In one embodiment there is disclosed a composition comprising, separately or together, a GIPR agonist and a GLP-2R agonist, such as a GIP peptide and a GLP-2 peptide, wherein said GIP peptide and said GLP-2 peptide are administered simultaneously, sequentially or separately

In one embodiment there is disclosed a composition comprising, separately or together, a GIPR agonist and a GLP-2R agonist, such as a GIP peptide and a GLP-2 peptide, wherein said composition is administered prior to sleep, such as once per day prior to sleep, such as in the evening prior to sleep, such as at bed time.

In one embodiment the composition is administered 1 to 120 minutes prior to sleep, such as 1-5, 5-10, 10-15, 15-20, 20-25, 25-30, 30-35, 35-40, 40-45, 45-50, 50-55, 55-60, 60-70, 70-80, 80-90, 90-100, 100-110, 110-120 minutes prior to sleep.

In one embodiment the composition is administered at around 8 p.m., around 8:30 p.m., around 9 p.m., around 9:30 p.m., around 10 p.m., around 10:30 p.m., around 11 p.m., around 11:30 p.m., around 12 p.m., around 12:30 a.m., or at around 1 a.m.

In one embodiment the composition is administered prior to sleep, such as once per day prior to sleep, such as in the evening prior to sleep, such as at bed time; and administered again after 2 hrs, such as 3 hrs, for example 4 hrs, such as 5 hrs, for example 6 hrs.

### Routes of Administration

It will be appreciated that the preferred route of administration will depend on the general condition and age of the subject to be treated, the nature of the condition to be treated, the location of the tissue to be treated in the body and the active ingredient chosen.

### Systemic Treatment

For systemic treatment the route of administration is capable of introducing a composition comprising, separately or together, a GIPR agonist and a GLP-2R agonist, such as a GIP peptide and a GLP-2 peptide, and optionally a GLP-1 peptide, into the blood stream to ultimately target the sites of desired action.

Such routes of administration are any suitable routes, such as an enteral route (including the oral, rectal, nasal, pulmonary, buccal, sublingual, transdermal, intracisternal and intraperitoneal administration), and/or a parenteral route (including subcutaneous, intramuscular, intrathecal, intracerebral, intravenous and intradermal administration).

In one embodiment the composition as disclosed herein is administered systemically.

### Parenteral Administration

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration or subcutaneous administration. Furthermore, parenteral administration includes inhalations and topical administration.

In one embodiment the composition as disclosed herein is administered parenterally, including subcutaneous, intramuscular, intrathecal, intracerebral, intravenous and intradermal administration.

In one embodiment the composition as disclosed herein is administered subcutaneously.

Accordingly, the present composition may be administered topically to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth, and accordingly, parenteral administration may also include buccal, sublingual, nasal, rectal, vaginal and intraperitoneal administration as well as pulmonal and bronchial administration by inhalation or installation. Also, the composition may be administered topically to cross the skin.

### Local Treatment

The composition comprising, separately or together, a GIPR agonist and a GLP-2R agonist, such as a GIP peptide and a GLP-2 peptide, and optionally a GLP-1 peptide, as defined herein may in one embodiment be used as a local treatment, i.e. be introduced directly to the site(s) of action. Accordingly, it may be applied to the skin or mucosa directly, or it may be injected into the site of action, for example into the diseased tissue or to an end artery leading directly to the diseased tissue.

Sequences

hGIP: YAEGTFISDYSIAMDKIHQQDFVNWLLAQKGKKNDWKHNITQ (SEQ ID NO: 1)
hGLP-2: HADGSFSDEMNTILDNLAARDFINWLIQTKITD (SEQ ID NO: 2)
hGLP-2-Arg: His-Ala-Asp-Gly-Ser-Phe-Ser-Asp-Glu-Met-Asn-Thr-Ile-Leu-Asp-Asn-Leu-
Ala-Ala-Arg-Asp-Phe-Ile-Asn-Trp-Leu-Ile-Gln-Thr-Lys-Ile-Thr-Asp-Arg (SEQ ID NO: 3)
hGIP(1-30): Tyr-Ala-Glu-Gly-Thr-Phe-Ile-Ser-Asp-Tyr-Ser-Ile-Ala-Met-Asp-Lys-Ile-His-
Gln-Gln-Asp-Phe-Val-Asn-Trp-Leu-Leu-Ala-Gln-Lys (SEQ ID NO: 4)
Human Gly2GLP-2: H-His-Gly-Asp-Gly-Ser-Phe-Ser-Asp-Glu-Met-Asn-Thr-Ile-Leu-
Asp-Asn-Leu-Ala-Ala-Arg-Asp-Phe-Ile-Asn-Trp-Leu-Ile-Gln-Thr-Lys-Ile-Thr-Asp-OH
(SEQ ID NO: 5)
HGDGSFSDEMNTILDNLAARDFINWLIETKITD (L-histidylglycyl-L-α-aspartylglycyl-L-
seryl-L-phenylalanyl-L-seryl-L-α-aspartyl-L-α-glutamyl-L-methionyl-L-asparaginyl-L-
threonyl-L-isoleucyl-L-leucyl-L-α-aspartyl-L-asparaginyl-L-leucyl-L-alanyl-L-alanyl-L-
arginyl-L-α-aspartyl-L-phenylalanyl-L-isoleucyl-L-asparaginyl-L-tryptophyl-L-leucyl-L-
isoleucyl-L-glutaminyl-L-threonyl-L-lysyl-L-isoleucyl-L-threonyl-L-aspartic acid) (SEQ ID
NO: 6)
hGLP-1(1-37): HDEFERHAEGTFTSDVSSYLEGQAAKEFIAWLVKGRG (SEQ ID NO: 7)
hGLP-1(7-36): HAEGTFTSDVSSYLEGQAAKEFIAWLVKGR (SEQ ID NO: 8)
hGLP-1(7-37): HAEGTFTSDVSSYLEGQAAKEFIAWLVKGRG (SEQ ID NO: 9)
hGLP-1(1-36): HDEFERHAEGTFTSDVSSYLEGQAAKEFIAWLVKGR (SEQ ID NO: 10)
hGLP-1(9-36): EGTFTSDVSSYLEGQAAKEFIAWLVKGR (SEQ ID NO: 11)
A7-hGLP-1(7-36): AAEGTFTSDVSSYLEGQAAKEFIAWLVKGR (SEQ ID NO: 12)
A10-hGLP-1(7-36): HAEATFTSDVSSYLEGQAAKEFIAWLVKGR (SEQ ID NO: 13)
hGIP(2-30): AEGTFISDYSIAMDKIHQQDFVNWLLAQK (SEQ ID NO: 14)
D-Ala2-hGIP: Y(D-Ala)EGTFISDYSIAMDKIHQQDFVNWLLAQKGKKNDWKHNITQ
(SEQ ID NO: 15)
Pro3-hGIP: YAPGTFISDYSIAMDKIHQQDFVNWLLAQKGKKNDWKHNITQ (SEQ ID
NO: 16)
D-Ala2-hGIP(1-30): Y(D-Ala)EGTFISDYSIAMDKIHQQDFVNWLLAQK (SEQ ID NO: 17)
hGIP(34-42); NDWKHNITQ (SEQ ID NO: 18)
mGIP: YAEGTFISDYSIAMDKIRQQDFVNWLLAQRGKKNDWKHNITQ (SEQ ID NO: 19)
rGIP: YAEGTFISDYSIAMDKIRQQDFVNWLLAQKGKKNDWKHNITQ (SEQ ID NO: 20)
mGIP(1-30): YAEGTFISDYSIAMDKIRQQDFVNWLLAQR (SEQ ID NO: 21)
rGIP(1-30): YAEGTFISDYSIAMDKIRQQDFVNWLLAQK (SEQ ID NO: 22)
(L17K, K30R)GLP-2 HADGSFSDEMNTILDNKAARDFINWLIQTRITD (SEQ ID NO: 23)
Ex4: HGEGTFTSDLSKQMEEEAVRLFIEWLKNGGPSSGAPPPS (SEQ ID NO: 24)
Ex4(1-30): HGEGTFTSDLSKQMEEEAVRLFIEWLKNGG (SEQ ID NO: 25)
Ex4(9-39) DLSKQMEEEAVRLFIEWLKNGGPSSGAPPPS (SEQ ID NO: 26)
Ex(9-30) DLSKQMEEEAVRLFIEWLKNGG (SEQ ID NO: 27)
Pro2-GIP(1-30): YPEGTFISDYSIAMDKIHQQDFVNWLLAQK (SEQ ID NO: 28)
Y(CH₂NH)-Glu3-GIP(1-30): YAEGTFISDYSIAMDKIHQQDFVNWLLAQK (SEQ ID
NO: 29)
(P)Ser2-GIP(1-30): Y(P)SEGTFISDYSIAMDKIHQQDFVNWLLAQK (SEQ ID NO: 30)
Val2-GIP(1-30): YVEGTFISDYSIAMDKIHQQDFVNWLLAQK (SEQ ID NO: 31)
Gly2-GIP(1-30): YGEGTFISDYSIAMDKIHQQDFVNWLLAQK (SEQ ID NO: 32)
Ser2-GIP(1-30): YSEGTFISDYSIAMDKIHQQDFVNWLLAQK (SEQ ID NO: 33)
D-Tyr1-GIP(1-30): (D)YAEGTFISDYSIAMDKIHQQDFVNWLLAQK (SEQ ID NO: 34)
D-Glu3-GIP(1-30): YA(D)EGTFISDYSIAMDKIHQQDFVNWLLAQK (SEQ ID NO: 35)

### EXAMPLES

### Example 1

### Aim:

Investigating the effect of subcutaneously administered native GIP and GLP-2 on bone remodelling.

### Method:

Eight healthy young men were enrolled. The study included three study days in randomised order (with a minimum of 1 week washout between study days) where human GIP (200 μg), human GLP-2 (800 μg), or placebo was injected subcutaneously. Participants arrived in the morning (fasted overnight) and received the injection around 8.30 a.m. Blood samples were collected before (−25 and −10 min) and every 30 minutes after injection (30, 60, 90, 120, 180 and 240 min). After last participant's last visit, bone resorption was determined by measurements of serum C-terminal cross-linking telopeptide of type I collagen (CTX; IDS Immunodiagnostic Systems GmbH, Frankfurt am Main, Germany)

### Results:

GIP (200 μg) and GLP-2 (800 μg) both inhibited bone resorption as measured by plasma CTX concentration. The maximum effect of GIP was seen 90 min after injection where bone resorption was reduced to 56.2±11.2% of base line. The maximum effect of GLP-2, down to 63.4±3.4% of base line was reached 180 min after administration (see FIG. 1).

### Conclusion:

These results show that administration of GIP and GLP-2 both inhibited bone resorption; furthermore a subcutaneous injection of GIP results in a marked reduction in bone resorption.

### Example 2

### Aim:

Proof of concept study investigating the synergistic effect of native GIP and GLP-2 on bone resorption by comparing the effects achieved when the hormones are administered alone with the effect reached when they are administered together.

### Method:

Ten healthy young men are enrolled and the study includes four study days in randomised order (with a minimum of 1 week washout between study days) where human GIP (100 μg), human GLP-2 (400 μg), GIP+GLP-2 (100 μg+400 μg), or placebo are injected subcutaneously. The participants arrive in the morning (fasted overnight) and receive the injection around 8.30 a.m. Blood samples are collected before (−25 and −10) and every 30 minutes after injection (30, 60, 90, 120, 180 and 240 min) injection. After last participant's last visit, samples are analysed for concentrations of bone resorption markers (CTX).

The doses of GIP and GLP-2 have been selected based on Example 1 showing pronounced (˜50%) reduction in bone resorption (CTX) which is comparable to the reduction seen after meal ingestion (which is believed to be the maximal achievable) using 200 μg GIP and 800 μg GLP-2. To be able to measure a synergistic/additive effect we have, therefore, reduced the doses in Example 1.

### Results:

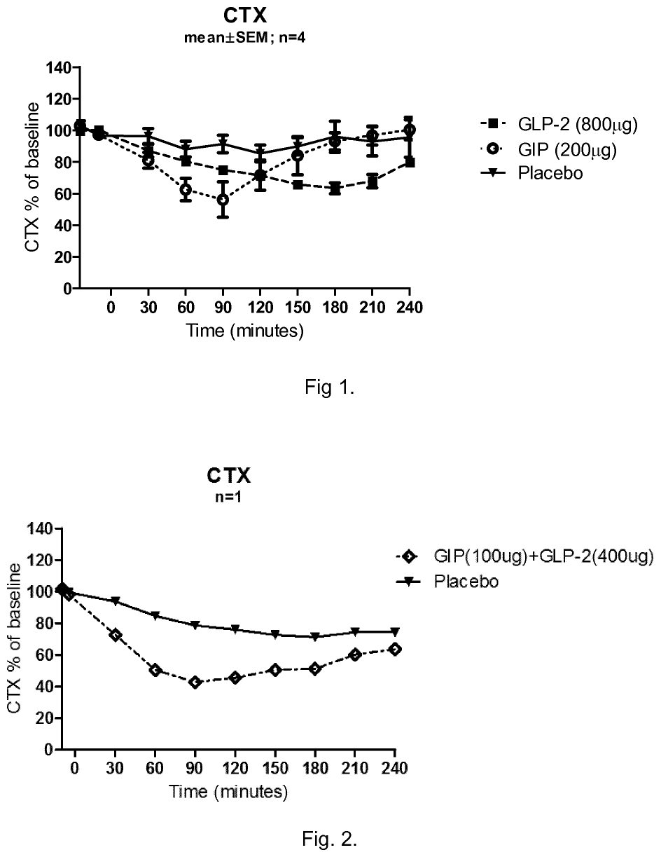

The combination of GIP and GLP-2 induced a pronounced reduction in bone resorption. The maximum effect was seen 90 min after injection where the bone resorption was reduced with approximately 60% (down to 42.8% of base line) (FIG. 2).

### Conclusion:

These results show that subcutaneous administration of GIP and GLP-2 together has a synergistic effect on reducing bone resorption.

### Example 3 (Completion of Example 1 Study)

### Aim:

Investigating the effect of subcutaneously administered native GIP and GLP-2 on bone remodelling.

### Method:

Eight healthy young men were enrolled, and the study was conducted as outlined in Example 1. After last participant's last visit, bone resorption was determined by measurements of CTX (IDS Immunodiagnostic Systems GmbH, Frankfurt am Main, Germany) and bone formation was determined by measurements of serum N-terminal propeptide of type 1 collagen (P1NP; IDS Immunodiagnostic Systems GmbH, Frankfurt am Main, Germany). In addition, serum concentration of parathyroid hormone (PTH; IDS Immunodiagnostic Systems GmbH, Frankfurt am Main, Germany) was measured.

### Results:

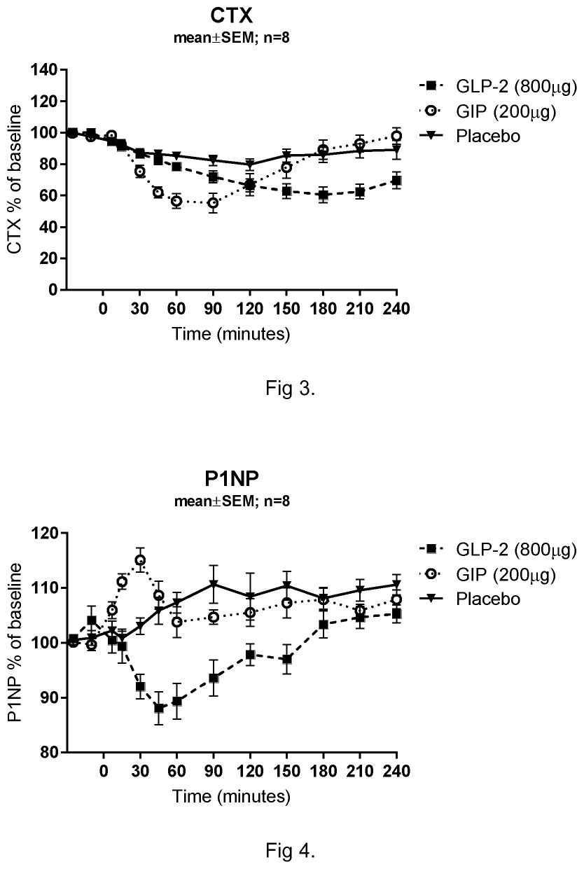

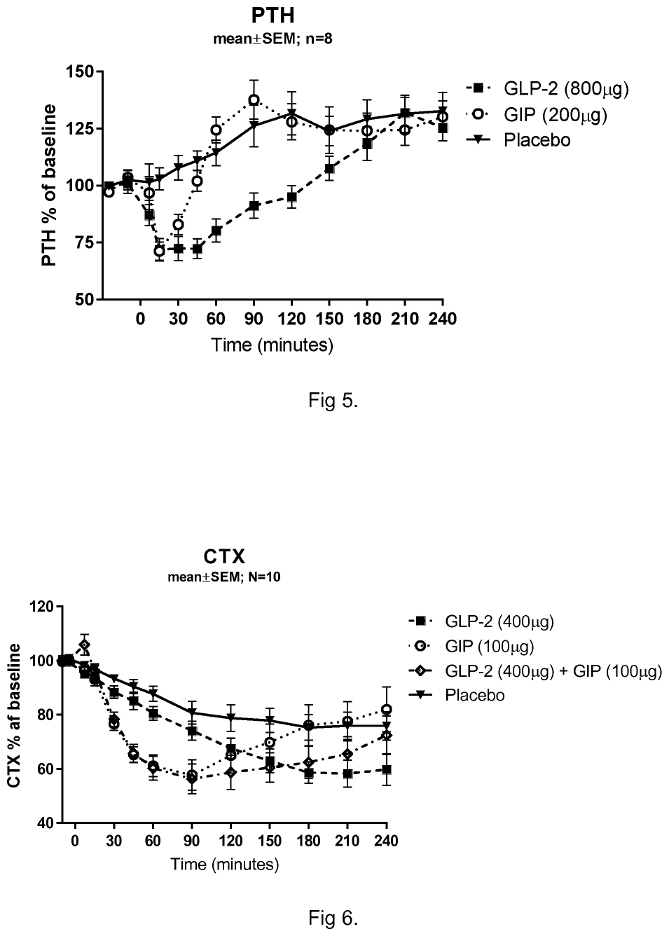

GIP (200 μg) and GLP-2 (800 μg) both inhibited bone resorption as measured by plasma CTX concentration. The maximum effect of GIP was seen 90 min after injection where bone resorption was reduced to 55.3±17.9% of base line. The maximum effect of GLP-2, down to 60.5±14.1% of base line was reached 180 min after administration (see FIG. 3). GLP-2 (800 μg) resulted in a reduction in bone formation (P1NP) with maximum effect 45 min after injection (reduced to 88.1±8.5% of base line), whereas GIP (200 μg) caused an increase in bone formation (P1NP) with maximum effect 30 min after subcutaneous injection (increased to 115.1±6.2% of base line) (see FIG. 4). Acute decreases in PTH levels were seen after GIP (200 μg) and GLP-2 (800 μg) administration with maximum effect reached 15 min after injection (reduced to 71.2±11.4% and 71.9±13.9% of basal levels, respectively) (see FIG. 5).

### Conclusion:

These results show that administration of GIP (200 μg) and GLP-2 (800 μg) inhibits bone resorption and reduces serum PTH levels. Furthermore, the data shows that GIP treatment increases bone formation.

### Example 4 (Completion of Example 2 Study)

### Aim:

Proof of concept study investigating the synergistic effect of native GIP and GLP-2 on bone resorption by comparing the effects achieved when the hormones are administered alone with the effect reached when they are administered together.

### Method:

Ten healthy young men are enrolled and the study was conducted as outlined in Example 2. After last participant's last visit, bone resorption was determined by measurements of CTX (IDS Immunodiagnostic Systems GmbH, Frankfurt am Main, Germany) and bone formation was determined by measurements of serum N-terminal propeptide of type 1 collagen (P1NP; IDS Immunodiagnostic Systems GmbH, Frankfurt am Main, Germany). In addition, serum concentration of parathyroid hormone (PTH; IDS Immunodiagnostic Systems GmbH, Frankfurt am Main, Germany) was measured. The doses of GIP and GLP-2 were selected as discussed in example 2.

### Results:

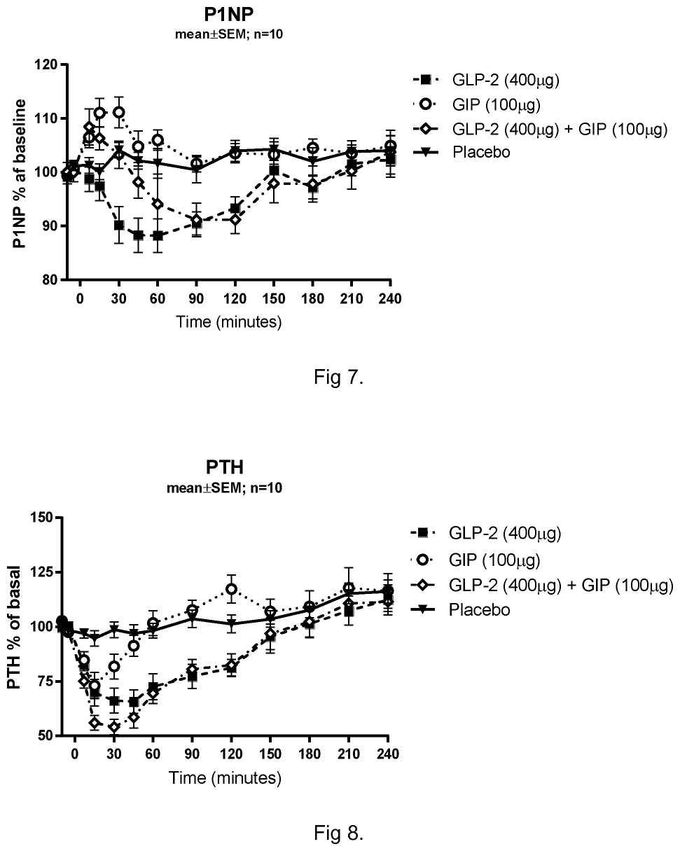

Analyses showed that GIP (100 μg) and GLP-2 (400 μg) injected alone or in combination in all cases resulted in decreases in bone resorption. For GIP alone and for GIP+GLP-2 the maximum effect (reduction to 57.6±17.9% and 56.2±17.4% of basal, respectively) was reached 90 min after administration. For GLP-2 the maximum effect (reduction to 58.2±16.0% of basal) was reached 210 min after injection (see FIG. 6). GLP-2 (400 μg) resulted in a reduction in bone formation (P1NP) with maximum effect 60 min after injection (reduced to 88.2±9.4% of base line), whereas GIP (100 μg) treatment caused an increase in bone formation (P1NP) with maximum effect 30 min after injection (increased to 111.1±9.0% of base line). Co-treatment with GIP (100 μg) and GLP-2 (400 μg) resulted in an acute increase in bone formation (to 108.4±10.5% of basal at 7 min) followed by a reduction (to 91.1±9.8% of basal) (see FIG. 7). Acute decreases in PTH levels were seen after GIP (100 μg) and GLP-2 (400 μg) treatment and also after GIP+GLP-2 co-treatment with maximum effect reached 15, 45 and 30 min after injection, respectively. The PTH levels were reduced to 73.0±19.2%, 65.7±16.8% and 54.1±11.1% of basal levels for GIP (100 μg), GLP-2 (400 μg) and GIP+GLP-2 co-treatment, respectively (see FIG. 8).

### Conclusion:

These results show that subcutaneous administration of GIP and GLP-2 together has a synergistic effect on reducing bone resorption and on reducing PTH.

### Example 5

### Aim:

Investigating the effect of subcutaneously administered GLP-1 on bone remodelling.

### Method:

Eight healthy young people were enrolled (five male, three female). The study included two study days in randomised order (with a minimum of 1 week washout between study days) where human GLP-1 (1.5 nmol/kg) or placebo was injected subcutaneously. Participants arrived in the morning after an overnight fast. Blood samples were collected before (−15 and 0 min) and after injection (15, 30, 45, 60, 90 and 120 min). After last participant's last visit, bone resorption was determined by measurements of serum C-terminal cross-linking telopeptide of type I collagen (CTX; IDS Immunodiagnostic Systems GmbH, Frankfurt am Main, Germany).

### Results:

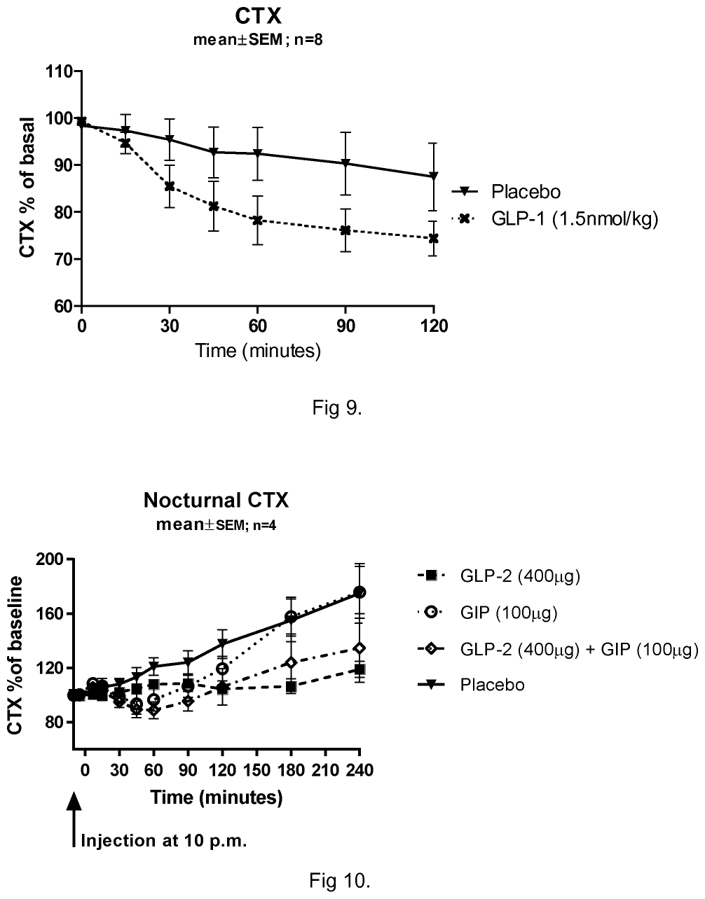

GLP-1 (1.5 nmol/kg) inhibited bone resorption as measured by plasma CTX concentration. The maximum effect of GLP-1 was seen 120 min after injection where bone resorption was reduced to 74.4±3.7% of base line. (see FIG. 9).

### Conclusion:

These data show that administration of GLP-1 inhibits bone resorption.

### Example 6

### Aim:

Investigating the effect of native GIP, GLP-2 and GIP+GLP-2 on nocturnal bone remodelling in postmenopausal women.

### Method:

Ten healthy postmenopausal women are enrolled and the study includes four study days in randomised order (with a minimum of 1 week washout between study days) where human GIP (hGIP 100 μg), human GLP-2 (hGIP 400 μg), hGIP+hGLP-2 (100 μg+400 μg), or placebo, are injected subcutaneously. The participants arrive in the evening around 9 p.m. (fasted from ˜7 p.m.) and receive the injections at 10 p.m. Blood samples are collected before (−10 and −5 min) and after injection (7, 15, 30, 45, 60, 90, 120, 180 and 240 min). Bone resorption was determined by measurements of serum C-terminal cross-linking telopeptide of type I collagen (CTX; IDS Immunodiagnostic Systems GmbH, Frankfurt am Main, Germany). The doses of GIP and GLP-2 were selected as discussed in example 2.

### Results:

Analyses showed that bone resorption increased continuously during the night on the placebo day as expected. On the placebo day CTX increased from 100% (base line) to 175±44%. After administration of GIP (100 μg) alone a reduced bone resorption was seen from 30 min to 120 min after injection compared to placebo. GLP-2 (400 μg) injection resulted in reduced bone resorption during the entire study period when compared to placebo. GIP+GLP-2 co-administration resulted in reduced bone resorption from 15 min to 240 min when compared to the placebo day (see FIG. 10).

### Conclusion:

These data show that GIP and GLP-2 administered alone or in combination inhibits the nocturnal increase in bone resorption in postmenopausal women. In addition, a synergistic effect on bone resorption is measured when GIP and GLP-2 are co-administered.

1. A composition comprising, separately or together, a GIPR agonist and a GLP-2R agonist, for use in a method of inhibiting bone resorption and/or stimulating bone formation.

2. A composition comprising, separately or together, a GIP peptide and a GLP-2 peptide, for use in a method of inhibiting bone resorption and/or stimulating bone formation.

3. A composition comprising, separately or together, a GIPR agonist and a GLP-2R agonist, for use in a method of treating a bone disorder.

4. A composition comprising, separately or together, a GIP peptide and a GLP-2 peptide, for use in a method of treating a bone disorder.

5. The composition for use according to any of the preceding claims, wherein said bone disorder is associated with poor or reduced bone density.

6. The composition for use according to any of the preceding claims, wherein said bone disorder is associated with increased bone resorption and/or reduced bone formation.

7. The composition for use according to any of the preceding claims, wherein said bone disorder is selected from the group consisting of osteopenia, osteoporosis, severe osteoporosis, osteomalacia, rickets, osteitis fibrosa cystica (OFC) and Paget's disease of bone.

8. The composition for use according to any of the preceding claims, wherein said bone disorder is osteopenia.

9. The composition for use according to any of the preceding claims, wherein said bone disorder is osteoporosis.

10. The composition for use according to any of the preceding claims, wherein said bone disorder is associated with a T-score of −1.0 or lower, such as between −1.0 and −2.5, such as −2.5 or lower.

11. The composition for use according to any of the preceding claims, wherein said treatment comprises one or more of treating, preventing and alleviating said bone disorder.

12. The composition for use according to any of the preceding claims, wherein said GIP peptide is hGIP (SEQ ID NO:1), or a functional variant or a functional fragment thereof.

13. The composition for use according to any of the preceding claims, wherein said GLP-2 peptide is hGLP-2 (SEQ ID NO:2), or a functional variant or a functional fragment thereof.

14. The composition for use according to any of the preceding claims, wherein said functional variant of a GIP peptide, such as SEQ ID NO:1, and said functional variant of a GLP-2 peptide, such as SEQ ID NO:2, has at least 60% sequence identity, such as at least 70% sequence identity, such as at least 75% sequence identity, such as at least 80% sequence identity, such as at least 85% sequence identity, such as at least 90% sequence identity, such as at least 95% sequence identity, such as at least 97% sequence identity to said GIP peptide, such as SEQ ID NO:1, and to said GLP-2 peptide, such as SEQ ID NO:2.

15. The composition for use according to any of the preceding claims, wherein said functional variant of said GIP peptide, and/or said functional variant of said GLP-2 peptide, comprises one amino acid substitution, two amino acid substitutions, three amino acid substitutions, four amino acid substitutions or five amino acid substitutions.

16. The composition for use according to any of the preceding claims, wherein said functional variant of said GIP peptide, and/or said functional variant of said GLP-2 peptide, comprises one or more conservative amino acid substitutions, such as one conservative amino acid substitution.

17. The composition for use according to any of the preceding claims, wherein said functional fragment of a GIP peptide, such as SEQ ID NO:1, comprises or consists of a consecutive stretch of amino acids of SEQ ID NO:1, or a variant thereof, said consecutive stretch comprising or consisting of 41 amino acids or less of SEQ ID NO:1, or a variant thereof, such as 10-15 amino acids, such as 15-20 amino acids, such as 20-25 amino acids, such as 25-30 amino acids, such as 30-35 amino acids, such as 35-41 amino acids of SEQ ID NO:1, or a variant thereof

18. The composition for use according to any of the preceding claims, wherein said functional fragment of GLP-2 peptide, such as SEQ ID NO:2, comprises or consists of a consecutive stretch of amino acids of SEQ ID NO:2, or a variant thereof, said consecutive stretch comprising or consisting of 32 amino acids or less of SEQ ID NO:2, or a variant thereof, such as 10-15 amino acids, such as 15-20 amino acids, such as 20-25 amino acids, such as 25-30 amino acids, such as 30-32 amino acids of SEQ ID NO:2, or a variant thereof.

19. The composition for use according to any of the preceding claims, wherein said GIP peptide is an analogue of hGIP; such as a protease-resistant analogue of hGIP.

20. The composition for use according to any of the preceding claims, wherein said GIP peptide is selected from the group consisting of hGIP(1-30) (SEQ ID NO:4); hGIP(2-30) (SEQ ID NO:14); D-Ala2-hGIP (SEQ ID NO:15); Pro3-hGIP (SEQ ID NO:16); D-Ala2-hGIP(1-30) (SEQ ID NO:17); hGIP(34-42) (SEQ ID NO:18); Pro2-GIP(1-30) (SEQ ID NO:28); γ(CH₂NH)-Glu3-GIP(1-30) (SEQ ID NO:29); (P)Ser2-GIP(1-30) (SEQ ID NO:30); Val2-GIP(1-30) (SEQ ID NO:31); Gly2-GIP(1-30) (SEQ ID NO:32); Ser2-GIP(1-30) (SEQ ID NO:33); D-Tyr1-GIP(1-30) (SEQ ID NO:34); D-Glu3-GIP(1-30) (SEQ ID NO:34); each optionally with a N-terminal H and/or a C-terminal —OH or —NH2; mGIP(1-30) (SEQ ID NO:21); rGIP(1-30) (SEQ ID NO:22); or a functional variant thereof.

21. The composition for use according to any of the preceding claims, wherein said GLP-2 peptide is an analogue of hGLP-2; such as a protease-resistant analogue of hGLP-2.

22. The composition for use according to any of the preceding claims, wherein said GLP-2 peptide is selected from the group consisting of teduglutide (Gattex; revestive); glepaglutide; SEQ ID NO:3 (hGLP-2 with an extra C-terminal Arg);Human Gly2GLP-2 (SEQ ID NO:5); SEQ ID NO:6; acylated versions of GLP-2 (aGLP-2); a GLP-2 analogue with two substitutions (SEQ ID NO:23) and the analogue of SEQ ID NO:23 acylated with a β-alanine spacer and a C16 fatty acid at the ε-amino group of Lys17 (cf. WO 2004/035624); or a functional variant thereof.

23. The composition for use according to any of the preceding claims, wherein said GIP peptide and/or said GLP-2 peptide is C-terminally amidated (—NH₂).

24. The composition for use according to any of the preceding claims, wherein said GIP peptide and/or said GLP-2 peptide is N-terminally acetylated (COCH₃).

25. The composition for use according to any of the preceding claims, wherein said functional variant and/or said functional fragment of GIP, such as of SEQ ID NO:1, is capable of one or more of:
a. binding to GIPR, and/or
b. activation of GIPR, and/or
c. stimulation of GIPR-activation, such as GIPR-mediated cAMP production, and/or
d. inhibiting bone resorption, and/or
e. stimulating bone formation.

26. The composition for use according to any of the preceding claims, wherein said functional variant and/or said functional fragment of GLP-2, such as of SEQ ID NO:2, is capable of one or more of:
a. binding to GLP2R, and/or
b. activation of GLP2R, and/or
c. stimulation of GLP2R-activation, such as GLP2R-mediated cAMP production, and/or
d. inhibiting bone resorption, and/or
e. stimulating bone formation.

27. The composition for use according to any of the preceding claims, wherein said GIP peptide is a full agonist of GIPR; and/or wherein said GLP-2 peptide is a full agonist of GLP-2R.

28. The composition for use according to any of the preceding claims, wherein said GIP peptide and said GLP-2 peptide are administered simultaneously, sequentially or separately.

29. The composition for use according to any of the preceding claims, wherein said composition further comprises, separately or together, a GLP-1R agonist, such as a GLP-1 peptide.

30. The composition for use according to claim 29, wherein said GLP-1 peptide is an analogue of hGLP-1, such as a protease-resistant analogue of hGLP-1.

31. The composition for use according to claims 29-30, wherein said GLP-1 peptide is selected from the group consisting of exenatide, lixisenatide, albiglutide, liraglutide, taspoglutide, dulaglutide, semaglutide, exendin-4 (Ex4; SEQ ID NO:24), Ex4(1-30) (SEQ ID NO:25), Ex4(9-39) (SEQ ID NO:26), Ex(9-30) (SEQ ID NO:27), SEQ ID NO:7 hGLP-1(1-37)), (SEQ ID NO:8 hGLP-1(7-36)), SEQ ID NO:9 (hGLP-1(7-37)), SEQ ID NO:10 (hGLP-1(1-36)), SEQ ID NO:11 (hGLP-1(9-36)), SEQ ID NO:12 (A7-hGLP-1(7-36)) and SEQ ID NO:13 (A10-hGLP-1(7-36)), or a functional variant thereof.

32. The composition for use according to any of the preceding claims, wherein said composition is administered systemically.

33. The composition for use according to any of the preceding claims, wherein said composition is administered parenterally, including subcutaneous, intramuscular, intrathecal, intracerebral, intravenous and intradermal administration.

34. The composition for use according to any of the preceding claims, wherein said composition is administered subcutaneously.

35. The composition for use according to any of the preceding claims, wherein said composition is administered prior to sleep, such as once per day prior to sleep, such as in the evening prior to sleep, such as at bed time.

36. The composition for use according to any of the preceding claims, wherein said composition is administered 1 to 120 minutes prior to sleep, such as 1-5, 5-10, 10-15, 15-20, 20-25, 25-30, 30-35, 35-40, 40-45, 45-50, 50-55, 55-60, 60-70, 70-80, 80-90, 90-100, 100-110, 110-120 minutes prior to sleep.

37. The composition for use according to any of the preceding claims, wherein said composition is administered at around 8 p.m., around 8:30 p.m., around 9 p.m., around 9:30 p.m., around 10 p.m., around 10:30 p.m., around 11 p.m., around 11:30 p.m., around 12 p.m., around 12:30 a.m., or at around 1 a.m.

38. The composition for use according to any of the preceding claims, wherein said composition is administered prior to sleep, such as once per day prior to sleep, such as in the evening prior to sleep, such as at bed time; and administered again after 2 hrs, such as 3 hrs, for example 4 hrs, such as 5 hrs, for example 6 hrs.

39. The composition for use according to any of the preceding claims, further comprising a further active pharmaceutical ingredient which is useful for treating a bone disorder, such as a bone disorder associated with reduced bone density.

40. The composition for use according to any of the preceding claims, further comprising a further active pharmaceutical ingredient selected from the group consisting of Bisphosphonates including Alendronate (Fosamax), Risedronate (Actonel, Atelvia, Benet), Ibandronate (Boniva), Zoledronic acid (Reclast, Aclasta, Zometa), Etidronic acid (Didronel), Pamidronic acid (Aredia/Pamimed), Tiludronic acid (Skelid); estrogen replacement therapy; hormone therapies; hormone-like medications including raloxifene (Evista); Calcitonin (Fortical and Miacalcin), Denosumab (Prolia); Teriparatide (Forteo); Vitamin D (alfacalcidol or calcitriol); and calcium or phosphorus supplement.

41. The composition for use according to any of the preceding claims, further comprising a further active pharmaceutical ingredient which is a DPP-4 inhibitor (Dipeptidyl Peptidase IV Inhibitor); such as Diprotin A; or a gliptin such as sitagliptin, saxagliptin, vildagliptin and alogliptin,
